(19) United States
(12) Patent Application Publication
(10) Pub. No.: US 2016/0346827 A1
(43) Pub. Date: Dec. 1, 2016

(54) Method of Fabricating a Surgical Needle with Recessed Features

(71) Applicant: Ethicon Endo-Surgery, LLC, Guaynabo, PR (US)

(72) Inventors: David T. Martin, Milford, OH (US); Frank R. Cichocki, JR., Easton, PA (US); Thomas Nering, Milford, NJ (US); James M. Dick, Flowery Branch, GA (US)

(21) Appl. No.: 14/721,251

(22) Filed: May 26, 2015

(51) Int. Cl. B21G 1/00; A61B 17/06

(52) U.S. Cl. CPC B21G 1/00; A61B 17/06066; A61B 2017/061; A61B 2017/06095; A61B 2017/00526

(57) ABSTRACT

A method is used for fabricating a surgical needle for use in with a circular needle applier having a needle driver. A wire body is straightened and cut to an initial length, the wire body having a first end and a second end. A point is ground on the first end of the wire body. The wire body is plastically deformed to form two recessed features adapted to be engaged by a needle driver. The wire body is cut to a final length with a trailing end. A hole is drilled in the trailing end to create a barrel. The wire body is bent along a circular arc while keeping straight a trailing portion with the barrel. A length of suture is attached in the barrel. The trailing portion is bent along the circular arc.

### BACKGROUND

The present invention relates in general to surgical devices and procedures, and more particularly to surgical suturing.

Sutures are often used in a wide variety of surgical procedures. Manual suturing is typically accomplished by the surgeon using a fine pair of graspers to grab and hold a suture needle, pierce the tissue with the needle, let go of the needle, and regrasp the needle to pull the needle and accompanying suture thread through the tissues to be sutured. Such needles are typically curved with the suture attached to the trailing end of the needle. A variety of automated suturing devices have been attempted to speed the process of suturing and to facilitate fine suturing or suturing during endoscopic, laparoscopic, or arthroscopic surgeries.

### BRIEF DESCRIPTION OF DRAWINGS

While the specification concludes with claims which particularly point out and distinctly claim the invention, it is believed the invention will be better understood from the following description taken in conjunction with the accompanying drawings illustrating some non-limiting examples of the invention. Unless otherwise indicated, the figures are not necessarily drawn to scale, but rather to illustrate the principles of the invention.

FIG. 1 depicts a side view of a surgical suturing device;

FIG. 2 depicts an exploded view of a cartridge;

FIG. 3 depicts a perspective view of a transmission for driving a needle;

FIG. 4 depicts a needle driver engaging a needle;

FIG. 5 depicts a needle body during a fabrication process;

FIG. 6 depicts a needle body during a fabrication process;

FIG. 7 depicts a needle body during a fabrication process;

FIG. 8 depicts a needle body during a fabrication process;

FIG. 8A depicts a cross-sectional view of a needle body from FIG. 8;

FIG. 9 depicts a needle body during a fabrication process;

FIG. 9A depicts a detailed view of a drive feature from FIG. 9;

FIG. 9B depicts a cross-sectional view of a needle body from FIG. 9A;

FIG. 10 depicts a needle body during a fabrication process;

FIG. 11 depicts a needle body during a fabrication process;

FIG. 12 depicts a needle body during a fabrication process;

FIG. 13 depicts a needle body during a fabrication process; and

FIG. 14 depicts a needle body during a fabrication process.

### SUMMARY

In one embodiment, a surgical needle is adapted for use with a circular needle applier having a needle driver. The surgical needle comprises an elongate body curved along a circular arc in a plane. The elongate body has a leading end, a trailing end, a longitudinal axis between the leading and trailing ends, a cross-sectional circumference, an upper face, a lower face, a medial face, and a lateral face. A pair of recessed features on the body are adapted to be engaged by a needle driver. A pair of protuberances are adjacent each recessed feature. The protuberances are longitudinally coincident with the respective recessed feature and circumferentially offset from the respective recessed feature. The protuberances project outwardly from the body.

Each recessed feature may comprise a step portion on the leading side that descends into a valley portion and a tiered surface rising from the trailing side of the valley portion. The tiered surface may comprise a flat portion and a pair of oblique portions, the flat portion being generally parallel with the longitudinal axis and intermediate the oblique portions. The apex of the protuberances may longitudinally coincide with the valley portion of the respective recessed feature.

The recessed features are on the medial face and the protuberances may be on the upper and lower faces. The pair of recessed features may be positioned at antipodal points on the circular arc. The surgical needle may further comprise a longitudinal flat on the body. The longitudinal flat may be intermediate the recessed features. The longitudinal flat and recessed features may be all circumferentially aligned. The protuberances may project about 3-10% the diameter of the body. The protuberances project about 6-8% the diameter of the body. The recessed features may be formed by a pressing operation. The recessed features may be formed without removing material from the body.

The surgical needle may further comprise a length of suture connected to the trailing end. A cartridge may comprise the surgical needle and a needle driver. The cartridge may further comprise a rotary input and a link connecting the needle driver to the rotary input.

In another embodiment, a surgical needle is adapted for use with a circular needle applier having a needle driver. The surgical needle comprises an elongate body curved along a circular arc in a plane. The elongate body has a leading end, a trailing end, a longitudinal axis between the leading and trailing ends, a cross-sectional circumference, an upper face, a lower face, a medial face, and a lateral face. A first recessed feature is on the medial face of the body adapted to be engaged by a needle driver. A first pair of protuberances project outwardly from the upper and lower faces of the body, the first pair of protuberances being longitudinally coincident with the first recessed feature. A second recessed feature is on the medial face of the body adapted to be engaged by a needle driver, the second recessed feature being located at the antipodal point of the circular arc relative the first recessed feature. A second pair of protuberances project outwardly from the upper and lower faces of the body, the second pair of protuberances being longitudinally coincident with the second recessed feature.

In yet another embodiment, a method is used for fabricating a surgical needle for use in with a circular needle applier having a needle driver. The method comprises the steps:

a) straightening and cutting a wire body to an initial length, the wire body having a first end and a second end;

b) grinding a point on the first end of the wire body;

c) plastically deforming the wire body to form two recessed features adapted to be engaged by a needle driver;

d) cutting the wire body to a final length with a trailing end;

e) drilling a hole in the trailing end to create a barrel;

f) bending the wire body along a circular arc while keeping straight a trailing portion with the barrel;

g) attaching a length of suture in the barrel; and

h) bending the trailing portion along the circular arc.

The steps may be performed sequentially as listed. The method may further comprise before step (c) the step of pressing one or more longitudinal flats on the wire body. The method may further comprise between steps (f) and (g) the step of cleaning the wire body. The method may further comprise between steps (f) and (g) the step of heat treating the wire body. The method may further comprise between steps (f) and (g) the step of electro-polishing the wire body. The method may further comprise between steps (f) and (g) the step of coating the wire body with silicone. During step (f), the recessed features may be oriented medially from the wire body. The method may further comprise prior to step (d) the step of bending a tail in the second end of the wire body.

### DETAILED DESCRIPTION

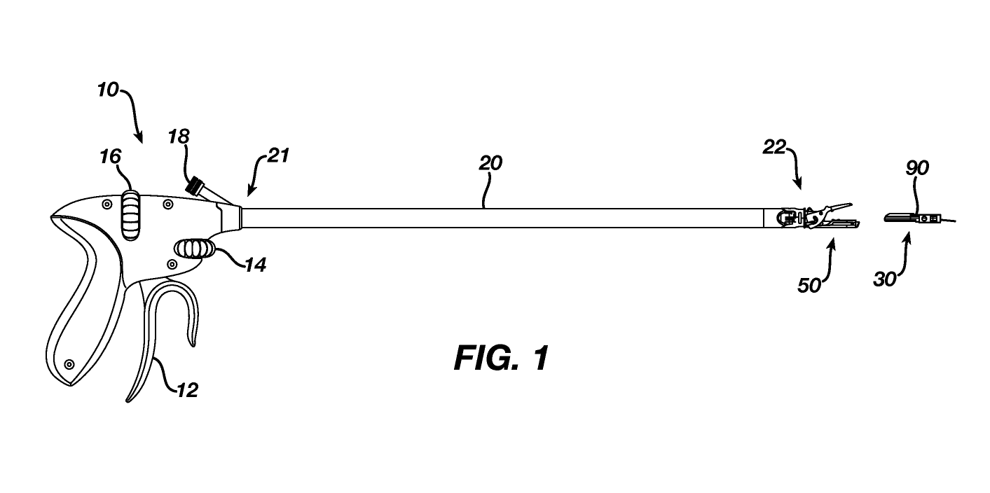

FIG. 1 illustrates an embodiment of a surgical suturing device. An elongate shaft (20) has a proximal end (21), a distal end (22), and a longitudinal axis extending therebetween. An actuator (10) is connected to the proximal end (21) of the shaft (20). In this embodiment the actuator (10) is a manual pistol grip handle; however, a variety of other manual actuators could also be used, including a scissor grip handle, a syringe grip handle, endoscopic rotary knobs, and the like. The actuator (10) could also take the form of a robotic interface, such as a DAVINCI puck, or a housing comprising gears or pulleys, servomechanisms, and the like.

A circular needle applier (30) is connected to the distal end (22) of the shaft (20). The circular needle applier (30) rotates an arced needle in a circular path enabling a surgeon to selectively apply sutures. The circular needle applier (30) may be integral with the shaft (20) and actuator (10) as a unitary disposable instrument intended for a single surgical procedure. The circular needle applier (30) may also be integral with the shaft (20) and actuator (10) as a reusable instrument. Optionally, as illustrated here, the circular needle applier (30) may be embodied in a disposable cartridge (90) and the shaft (20) may include a receiver (50) to hold the cartridge (90). In such an embodiment, the shaft (20) and actuator (10) may also be disposable or reusable. Embodiments with reusable components are intended to be cleaned, sterilized, and reused for a multiple surgical procedures, and may include a flush port (18) to facilitate cleaning The preferable life cycle of a reusable instrument is at least 50 operations, more preferably at least 150 operations, and most preferably at least 200 operations. Reusable components may be built using materials that can withstand autoclave sterilization temperatures of at least 135 degrees Celsius, although low temperature materials can also used with low temperature sterilization techniques known in the art.

A first input (12), shown here as a trigger that pivots between opened and closed positions, may be used to selectively actuate the circular needle applier (30). The trigger may be spring biased to return the trigger to its open position. A second input (14), shown here as a rotary knob, may be used to selectively articulate the shaft (20). A third input (16), shown here as a rotary knob, may be used to selectively rotate the circular needle applier (30) about the shaft (20). Naturally, the number, type, configuration, and operation of the inputs (12, 14, and 16) may vary.

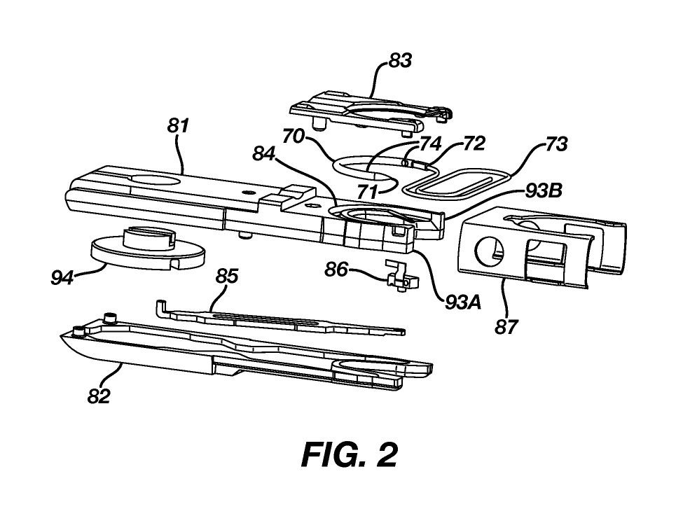

FIG. 2 illustrates an example of a cartridge (90) comprising a lower body (81), an upper body (82), and a needle cover (83). The needle driver (86), rotary input (94), and link (85) are captured between the lower body (81) and an upper body (82). The lower and upper bodies (81, 82) are attached to one another using a variety of known techniques, including welds, pins, adhesives, and the like to form the cartridge body. The needle (70) has a leading end (71) and a length of suture (73) extending from the trailing end (72). The needle (70) rotates in a circular path defined by the needle track (84) and between the arms (93A, B). Recessed features (74) may be provided to facilitate the needle driver (86) to engage and drive the needle (70).The needle (70) is captured in the needle track (84) by the needle cover (83). The cage (87) slides over the cartridge body to attach the needle cover (83) against the lower body (81).

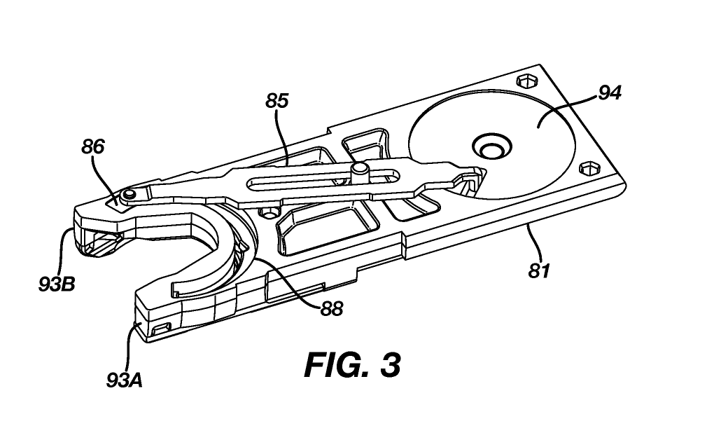

FIG. 3 illustrates an embodiment of a drive stroke of the transmission in the cartridge (90) for driving a needle (70) in a circular path. The needle driver (86) rides in the carrier track (88) and extends into the needle track (84) to engage and drive the needle (70). The link (85) connects the rotary input (94) to the needle driver (86). Counterclockwise rotation of the rotary input (94) will translate the needle driver (86) clockwise along the carrier track (88) driving the needle (70) clockwise until it reaches the other end of its stroke in the carrier track (88). In this embodiment, the drive stroke rotates the needle (70) in its circular path about 180 degrees. For the return stroke, the sequence can be reversed by rotating the rotary input (94) clockwise, which will translate the needle driver (86) counterclockwise in the carrier track (88) while the needle (70) remains stationary. Thus, a sequence of drive and return strokes will rotate the needle (70) in a circular path.

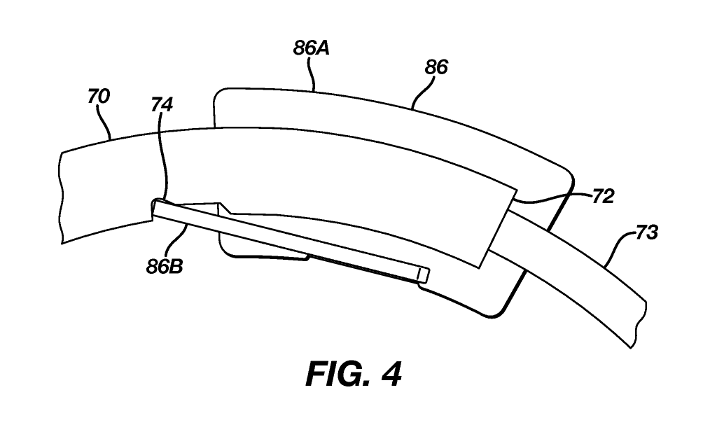

FIG. 4 illustrates a detailed view of the needle driver (86) engaging the needle (70). The needle driver (86) comprises a carrier (86A) and a driver (86B). The carrier (86A) is dimensioned to slideably fit in the carrier track (88). The driver (86B) is attached to the carrier (75) and is operative to engage the needle (70) at an oblique angle. Leftward movement of the needle driver (86) will cause the driver (86B) to engage the feature (74) during the drive stroke. When so engaged, the needle (70) will slide in the needle track (84) in unison with the needle driver (86). Due to the oblique angle, rightward movement of the needle driver (86) will disengage the driver (86B) from the feature (74) and slide over the stationary needle (70) during the return stroke.

Further details, explanations, examples, and alternative embodiments of surgical suturing devices and subcomponents of the foregoing are disclosed in co-owned U.S. application Ser. No. 13/832595 filed 15 Mar. 2013 (docket number END7266USNP) and application Ser. No. 14/297993 filed 6 Jun. 2014 (docket number END7501USNP). The foregoing applications are incorporated herein by reference.

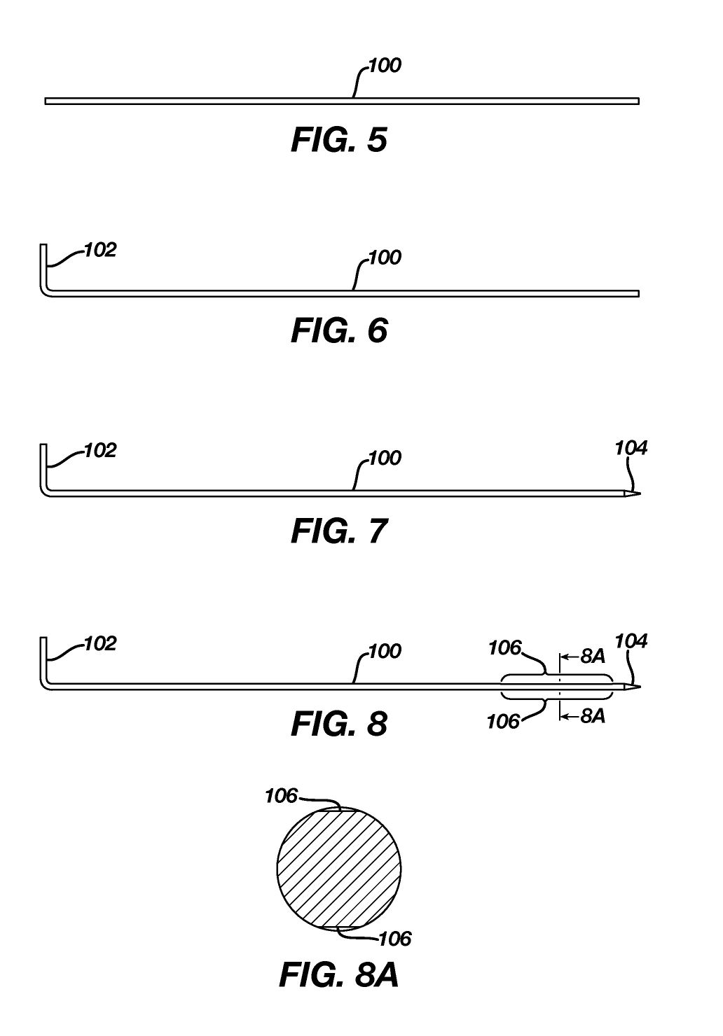

FIGS. 5-14 illustrate a method to fabricate the needle (70). Wire is fed off a spool, straightened, and cut to an initial length as shown in FIG. 5 to define an elongate body (100) having a longitudinal axis and a cross-sectional circumference. Many suitable wire materials may be used and may have a variety of different sizes and cross-sectional shapes. A typical wire may have a cross-sectional area between about 3×10⁻⁵ to about 0.005 in². In the embodiment depicted in FIG. 5, the wire is a stainless steel alloy having a circular cross-section with a nominal diameter of about 0.029 inches and a body (100) length of about 3 inches.

In FIG. 6, a tail (102) is formed on the trailing end of the body (100) for clocking and holding the body (100) during the fabrication process. In this embodiment, the tail (102) is bent generally normal to the body (100), but oblique or other tail shapes are also contemplated.

In FIG. 7, the point (104) is ground on the leading end of the body (100) to the desired shape for the leading end (71) of the needle (70).

In FIGS. 8 and 8A, one or more flats (106) are pressed on body (100). The flats (106) may serve as reference or indexing surface as the body is processed during subsequent steps. The flats (106) may also serve as a reference or indexing surface to hold the needle (70) square within the needle track (84). In this embodiment, two longitudinal flats (106) are arranged at antipodal locations, with the top one flat (106) being circumferentially aligned with the tail (102). In this embodiment, the flats (106) are about equal in length and longitudinally coextensive. Other configurations of the flats (106) are also contemplated.

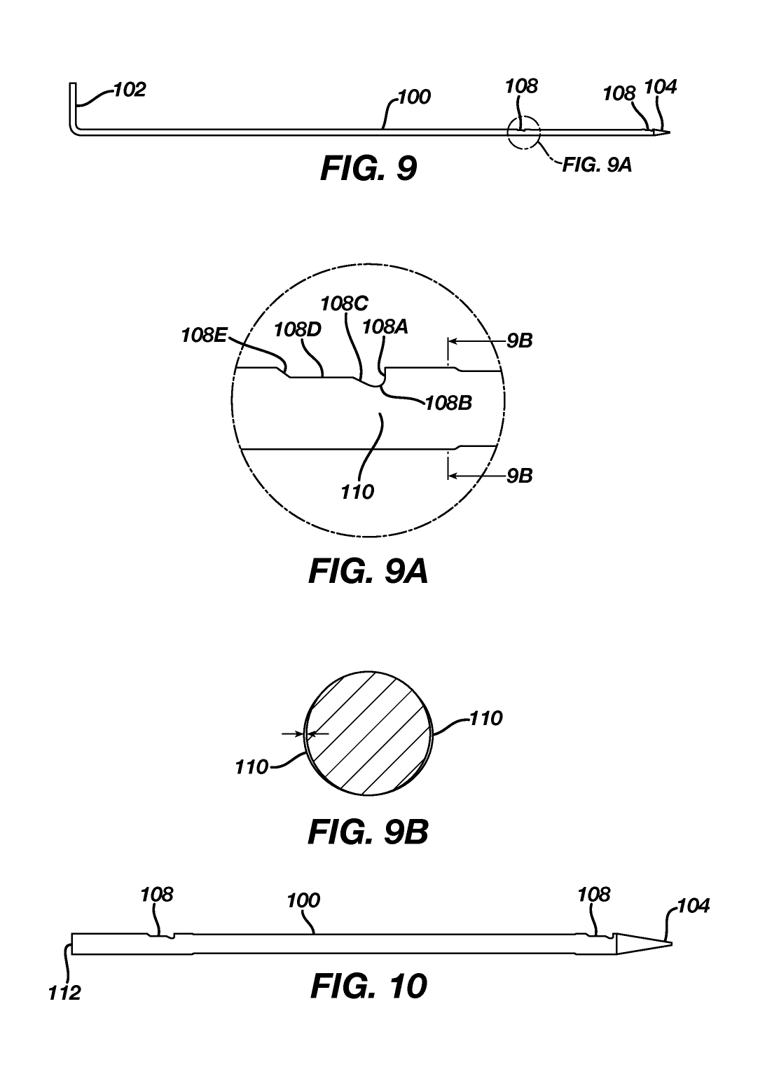

In FIGS. 9 and 9A-B, a plurality of recessed features (108) are formed into the body (100). In this embodiment two features (108) are formed at locations longitudinally spaced from the flats (106) such that the flats are interposed between the features (108). In this embodiment, the features (108) are circumferentially aligned with the top flat (106) and the tail (102). In this embodiment, the leading side of each feature (108) has a step portion (108A) that descends into a valley portion (108B). The step portion (108A) is transverse the longitudinal axis. The depth of the valley portion (108B) is preferably about 5 to 35% the diameter of the body (100) (about 0.003 to 0.009 inches in this example), and more preferably about 15 to 25%. A tiered surface defined by portions (108C-E) rises from the trailing side of the valley portion (108B). Oblique portions (108C, 108E) are arranged at an oblique angle relative the longitudinal axis of the body (100). Flat portion (108D) is intermediate the oblique portions (108C, 108E) and is generally parallel with the longitudinal axis of the body (100). The configuration of the flat portion (108D) and oblique portions (108C, 108E) facilitate a smooth transition of the driver (86B) from the valley portion (108B). Preferably, portions (108A-E) transition to one another with radiuses greater than about 0.002 inches to reduce stress concentrations and to reduce wear on tooling.

In this embodiment the features (108) are formed by plastically deforming the body (100). For instance, a die may be used in a pressing or rolling operation on the body (100) to form the features (108). A plastically deforming operation offers several advantages over techniques involving the removal of material, such as cutting or grinding operations. First, the cross-sectional area will remain substantially the same along the length of the body (100) around the feature (108) resulting in improved strength. Second, plastically formed features (108) are more reliable and reproducible, and capable of faster production. Third, material will be displaced away from the feature (108) resulting in a pair of protuberances (110) projecting outwardly from the body (100). The protuberances (110) are adjacent to and circumferentially offset on either side of the feature (108), and longitudinally coincide with the feature (108). Preferably, the apex of the protuberances (110) longitudinally coincide with the valley portion (108B). The protuberances (110) help center the needle (70) in the track (84), thus facilitating proper alignment of the needle (70) during the drive and return strokes. The height on the protuberances (110) will vary depending upon the gauge of the body (100) and the depth of the feature (108), but each protuberance (110) preferably projects outward about 3-10% the diameter of the body (100) (about 0.001-0.003 inches in this example), and more preferably about 6-8%, as generally shown by the arrows in FIG. 9B. Finally, the spacing of the features (108) may be made very precisely and reproducibly using a single piece die, so the location of features (108) will remain identically spaced from needle-to-needle over the life span of the tooling.

In FIG. 10, the body (100) is cut at the trailing end (112) to the final length of the needle (70).

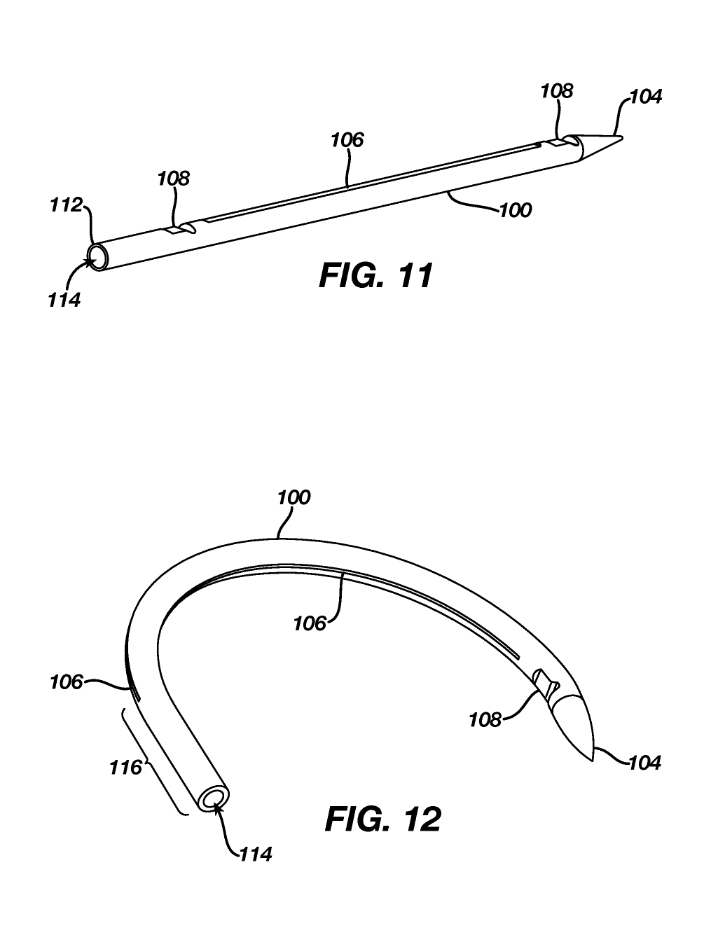

In FIG. 11, a hole is drilled in the trailing end (112) to create a barrel (114).

In FIG. 12, the body (100) is bent to the desired arcuate shape. In this embodiment, the body (100) is curved along a circular path; however, the trailing portion (116) with the barrel (114) is kept straight. The diameter of the circular arc may vary based on the desired size of the needle (70), but in this embodiment the centerline radius of curvature is about 0.2 inches. The curved body (100) defines a plane dividing upper and lower faces on the body (100). The flats (106) are positioned on the medial and lateral faces of the body (100), with the features (108) oriented on the medial face and the protuberances projecting outwardly from the upper and lower faces of the body (100). The flats (106) may be referenced prior to bending to facilitate the desired orientation of the features (108). It should be appreciated, however, that the orientation of the features (108) may be changed (e.g., on the upper, lower, or lateral faces) depending upon where the needle driver (86) is intended to engage the needle (70). The body (100) may be cleaned, heat treated, electro-polished, and/or coated with a lubricious silicone. The silicone chemistry may be characterized as a polydimethylsiloxane with functionalized end groups to provide a combination of lubricity and multiple pass performance.

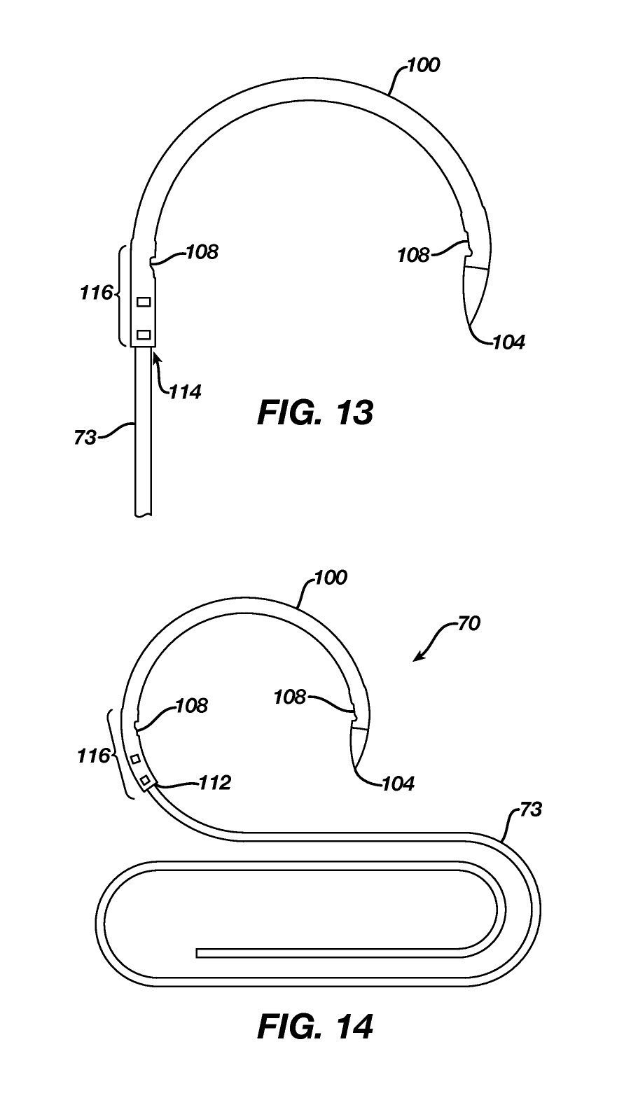

In FIG. 13, suture (73) is inserted into the barrel (114) and fixed to the body (100) with a swaging operation; however, other fixation techniques may also be used such as welding, adhesives, etc.

In FIG. 14, the trailing portion (116) is bent along the same circular arc as the remainder of the body (100), thus completing a fabricated needle (70). The features (108) are positioned at antipodal points on the circular arc, and the angular span between the point (104) and trailing end (112) is about 225 to 250 degrees.

Having shown and described various embodiments and examples of the present invention, further adaptations of the methods and devices described herein can be accomplished by appropriate modifications by one of ordinary skill in the art without departing from the scope of the present invention. Several of such potential modifications have been mentioned, and others will be apparent to those skilled in the art. For instance, the specific materials, dimensions, and the scale of drawings will be understood to be non-limiting examples. Similarly, some steps may be eliminated or performed in an alternative sequence. Accordingly, the scope of the present invention should be considered in terms of the following claims and is understood not to be limited to the details of structure, materials, or acts shown and described in the specification and drawings.

1. A method for fabricating a surgical needle for use in with a circular needle applier having a needle driver, comprising the steps:
a) straightening and cutting a wire body to an initial length, the wire body having a first end and a second end;
b) grinding a point on the first end of the wire body;
c) plastically deforming the wire body to form two recessed features adapted to be engaged by a needle driver;
d) cutting the wire body to a final length with a trailing end;
e) drilling a hole in the trailing end to create a barrel;
f) bending the wire body along a circular arc while keeping straight a trailing portion with the barrel;
g) attaching a length of suture in the barrel; and
h) bending the trailing portion along the circular arc.

2. The method of claim 1, wherein the steps are performed in a sequence as listed.

3. The method of claim 2, further comprising before step (c) the step of pressing one or more longitudinal flats on the wire body.

4. The method of claim 2, further comprising between steps (f) and (g) the step of cleaning the wire body.

5. The method of claim 2, further comprising between steps (f) and (g) the step of heat treating the wire body.

6. The method of claim 2, further comprising between steps (f) and (g) the step of electro-polishing the wire body.

7. The method of claim 2, further comprising between steps (f) and (g) the step of coating the wire body with silicone.

8. The method of claim 1, wherein during step (f) the recessed features are oriented medially from the wire body.

9. The method of claim 2, further comprising prior to step (d) the step of bending a tail in the second end of the wire body.
